(19) United States
(12) Patent Application Publication
(10) Pub. No.: US 2020/0254204 A1
(43) Pub. Date: Aug. 13, 2020

(54) FLEXIBLE ARTICULATING INTUBATION TOOL

(71) Applicants: Thomas Brian Moffat, Hugo, MN (US); Jack Ruegsegger, Ham Lake, MN (US)

(72) Inventors: Thomas Brian Moffat, Hugo, MN (US); Jack Ruegsegger, Ham Lake, MN (US)

(21) Appl. No.: 16/744,160

(22) Filed: Jan. 15, 2020

Related U.S. Application Data
(60) Provisional application No. 62/792,777, filed on Jan. 15, 2019.

(51) Int. Cl. A61M 16/04

(52) U.S. Cl. CPC A61M 16/0488; A61M 25/0136; A61M 16/0418

(57) ABSTRACT

A laryngoscopy tool for assisting in placement of an endotracheal tube is disclosed. The laryngoscopy tool includes a stylet having a proximal end and a distal end, the stylet configured for receiving an endotracheal tube, and having an articulating distal end, the articulating distal end being configured to articulate between more than one shape; a handle secured to the proximal end of the stylet.

### FIELD

The present application is directed to a tool for aiding in laryngoscopy, in particular a tool for assisting in placement of an endotracheal tube into a trachea.

### BACKGROUND

Approximately 8% of the population has partial to zero visual laryngeal exposure resulting in difficult laryngoscopy. In many instances, this partial view caused by anatomical variances inhibits placement of an endotracheal tube into the trachea. This inhibition is often due to the inability to manipulate the end of the rigid stylet and (superimposed endotracheal tube) while performing laryngoscopy. The spatial limitations imposed allow only modest movements of the stylet/endotracheal tube. Anatomical variances further inhibit the visualization of the trachea mandating precise control of the stylet/endotracheal tube for successful intubation of the trachea.

Therefore, a need exists for a means to aid in placement of an endotracheal tube into a trachea.

### SUMMARY OF THE INVENTION

The present disclosure is directed to a laryngoscopy tool for assisting in placement of an endotracheal tube. The laryngoscopy tool comprises a stylet having a proximal end and a distal end, the stylet configured for receiving an endotracheal tube. The stylet has an articulating distal end, the articulating distal end being configured to articulate between more than one shape. The tool also includes a handle secured to the proximal end of the stylet.

In an embodiment the handle is removable from the proximal end of the stylet.

In an embodiment the handle is removable from the proximal end of the stylet to allow placement of an endocardial tube in place.

In an embodiment the stylet articulates in a plane.

In an embodiment the stylet includes an internal mechanism for articulating the distal end of the stylet.

In an embodiment the stylet further comprises a flexible intermediate portion.

In an embodiment the stylet comprises an internal wire connected to the distal end, and wherein pulling on the wire from the proximal end results in articulating the distal end of the stylet.

In an embodiment the tool further comprising an actuator.

In an embodiment the actuator comprises a tube surrounding a portion of the proximal end of the stylet.

The present disclosure is also directed to a method for placement of an endotracheal tube, the method comprising: providing a laryngoscopy tool for assisting in placement of an endotracheal tube, the laryngoscopy tool including a stylet having a proximal end and a distal end, the stylet configured for receiving an endotracheal tube, and having an articulating distal end, the articulating distal end being configured to articulate between more than one shape. A handle is secured to the proximal end of the stylet. The method further includes inserting the distal end of the stylet of the laryngoscopy tool into the trachea of a person, articulating the distal end of the stylet during insertion to aid in passage through the larynx; removing the handle from the proximal end of the stylet; inserting an endotracheal tube over the proximal end of the stylet and down toward the distal end of the stylet; and removing the stylet from the patient's trachea will keeping the endotracheal tube in place.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a drawing showing a laryngoscopy tool for assisting in placement of an endotracheal tube made in accordance with an example embodiment.

FIG. 2A is a drawing showing a top view of a foreshortened laryngoscopy tool for assisting in placement of an endotracheal tube made in accordance with an example embodiment.

FIG. 2B is a drawing showing a side view of a foreshortened laryngoscopy tool for assisting in placement of an endotracheal tube made in accordance with an example embodiment.

FIG. 3 is a drawing showing a top view of a foreshortened laryngoscopy tool for assisting in placement of an endotracheal tube made in accordance with an example embodiment after removal of the handle.

FIG. 4A is a drawing showing a cross sectional side view of a foreshortened laryngoscopy tool for assisting in placement of an endotracheal tube made in accordance with an example embodiment, showing the distal end articulated.

FIG. 4B is a drawing showing a cross sectional side view of a foreshortened laryngoscopy tool for assisting in placement of an endotracheal tube made in accordance with an example embodiment, showing the distal end articulated.

FIG. 5 is a drawing showing a top view of a foreshortened laryngoscopy tool for assisting in placement of an endotracheal tube made in accordance with an alternative example embodiment.

FIG. 6 is a drawing showing a laryngoscopy tool for assisting in placement of an endotracheal tube made in accordance with an example embodiment.

FIG. 7 is a drawing showing a portion of a laryngoscopy tool for assisting in placement of an endotracheal tube made in accordance with an example embodiment.

FIG. 8 is a drawing showing a transition segment of a laryngoscopy tool that can transition from a stiffer main shaft to the more flexible articulation zone.

FIG. 9 is a partially-transparent image showing a transition segment of a laryngoscopy tool of FIG. 8 that can transition from a stiffer main shaft to the more flexible articulation zone.

FIG. 10 is a partially-transparent image showing a further section of a laryngoscopy tool of FIG. 8.

FIG. 11 is a partial cross section showing a further section of a laryngoscopy tool of FIG. 8.

FIG. 12 is an example tip of a laryngoscopy tool.

While embodiments are susceptible to various modifications and alternative forms, specifics thereof have been shown by way of example and drawings and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

The present disclosure is directed to a tool to aide in direct or video assisted laryngoscopy. The tool allows for articulation of the distal end of the stylet in one plane via a single hand operated controller on the proximal end of the stylet. This articulation provides increased range of motion so as to facilitate intubation where anatomical variances are present. The handle can be detached from the stylet allowing for intubation with or without endotracheal tube superimposed over the stylet. An alternative embodiment has the handle and controller integrated and detachable

In certain implementations a relatively flaccid middle portion of the stylet allows for easier entry and navigation of the pharynx due to minimal resistance of passive movement of stylet around anatomical structure.

The flaccid portion of the stylet allows for easier storage in code boxes and anesthesia carts. The device provides direct laryngoscopy compatibility. Unlike the rigid articulating stylets that require video optics to function optimally, the flaccid nature of this tool allows for better handling ergonomics when used under direct laryngoscopy.

The device can include a detachable handle, which allows the stylet to be placed into the trachea with or without the endotracheal tube in place. This provides maximal space and movement of the stylet in difficult anatomy to facilitate tracheal intubation. Once placed, the handle can be detached allowing for endotracheal tube to be placed over the stylet and slid down into the trachea. The stylet is then removed from the endotracheal tube.

In certain implementations the handle is designed to fit in the palm of hand so as to allow more precise control of stylet while under full wrist rotation. The handle also serves to provide a fulcrum for trigger engagement.

The device includes an activation trigger (actuator), which can be (for example) a round, knurled sheath wrapped around the stylet that is in close proximity to handle so as to allow thumb and finger activation. Its circular design allows for constant contact by thumb and fingers regardless of stylet rotation.

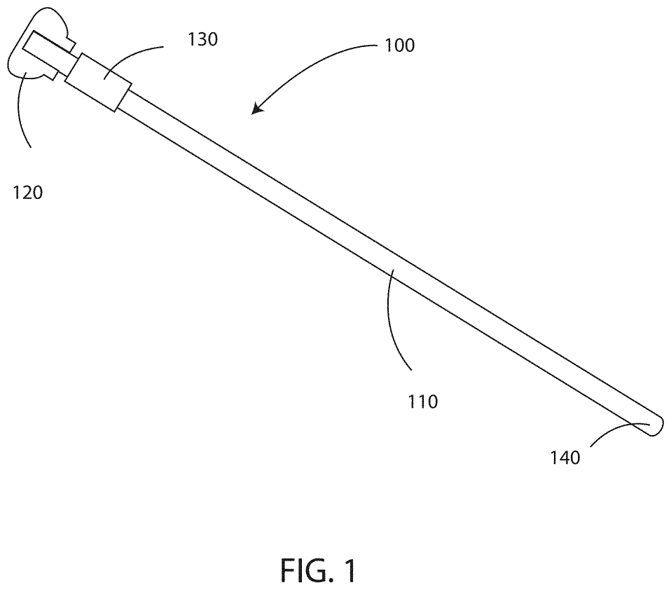

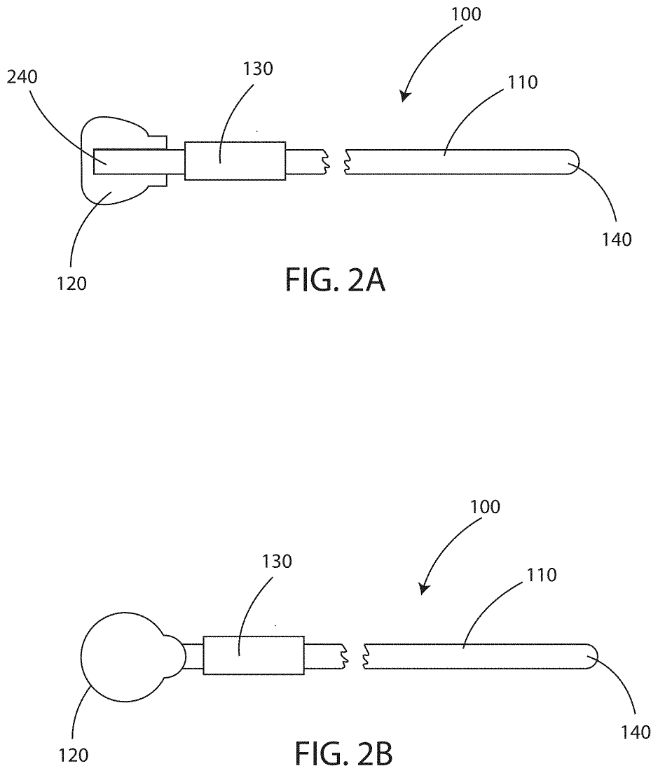

Referring now to the drawings, FIG. 1 is a drawing showing a laryngoscopy tool 100 for assisting in placement of an endotracheal tube made in accordance with an example embodiment. The laryngoscopy tool 100 includes a stylet 110, a handle 120 (at the proximal end of the tool), an actuator 130 and a distal end 140 at a distal end. The actuator 130 can be moved (such as sliding forward or backward, or by rotation) to engage an internal mechanism that causes the distal end 140 of the stylet 110 to bend (articulate). FIG. 2A is a drawing showing a top view of a laryngoscopy tool 100 of FIG. 1 that is foreshortened. The proximal end 240 of the stylet is shown, along with handle 120 (at proximal end of the tool), an actuator 130 and a distal end 140 at a distal end. The actuator 130 can be moved (such as sliding forward or backward, or by rotation) to engage an internal mechanism that causes the distal end 140 of the stylet 110 to bend. FIG. 2B is a drawing showing a side view of the laryngoscopy tool 100 for assisting in placement of an endotracheal tube made in accordance with an example embodiment of FIG. 2A.

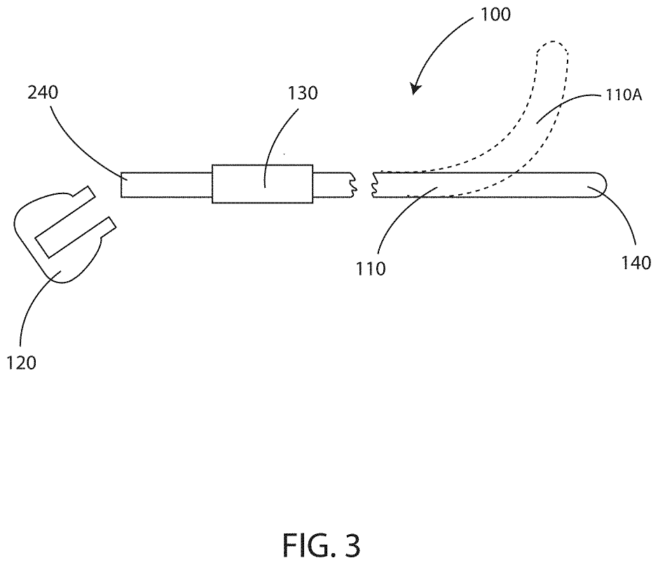

FIG. 3 is a drawing showing a top view of a foreshortened laryngoscopy tool for assisting in placement of an endotracheal tube made in accordance with an example embodiment after removal of the handle 120. Removal of handle 120 typically occurs when the stylet 110 is within the trachea of a patient, and removal of the handle allows for passage of an endotracheal tube (not shown) over the proximal end 240 of the stylet 110 and then down toward the distal end 140 of the stylet 110 (after which the stylet is withdrawn, leaving the endotracheal tube in place). FIG. 3 shows the stylet also in a bent embodiment, such as with about a 90 degree angle, represented in dashed lines as stylet 110A (which is stylet 110 but after having been bent). It will be appreciated that in typical use the angle can be varied, often from 90 degrees (straight) up to and 90 degrees.

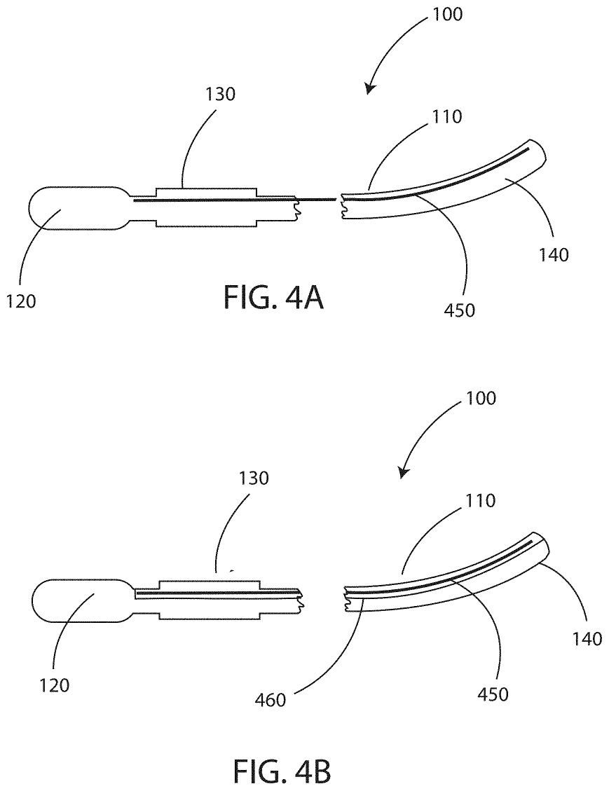

FIG. 4A is a drawing showing a cross sectional side view of a foreshortened laryngoscopy tool for assisting in placement of an endotracheal tube made in accordance with an example embodiment, showing the distal end articulated, showing an wire 450 in simplified form the wire in communication with the distal end 140 of the stylet 110 as well as the actuator 130 such that engagement of the actuator (such as by sliding it along the stylet 110 or rotating it) cause the wire 450 to move and articulate the distal end 140 of the stylet 110. FIG. 4B is a drawing showing a cross sectional side view of a foreshortened laryngoscopy tool for assisting in placement of an endotracheal tube made in accordance with an example embodiment, showing the distal end articulated, with the wire 450 within an interior lumen 460.

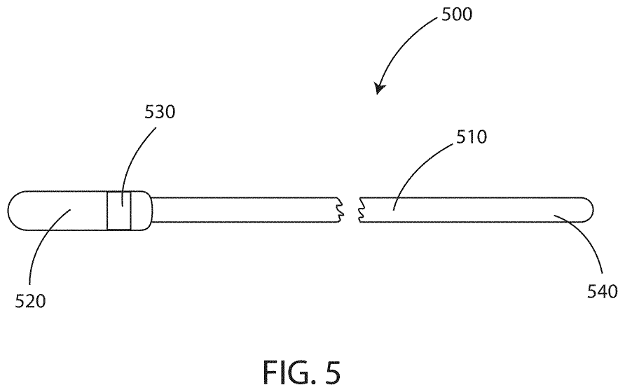

FIG. 5 is a drawing showing a top view of a laryngoscopy tool 500 for assisting in placement of an endotracheal tube made in accordance with an alternative example embodiment, the laryngoscopy tool 500 including a stylet 510, a handle 520, an actuator 530, and a tip 540. In this construction the handle 520 and actuator 530 are integrally formed as one piece. Also, in this construction the handle is substantially cylindrical.

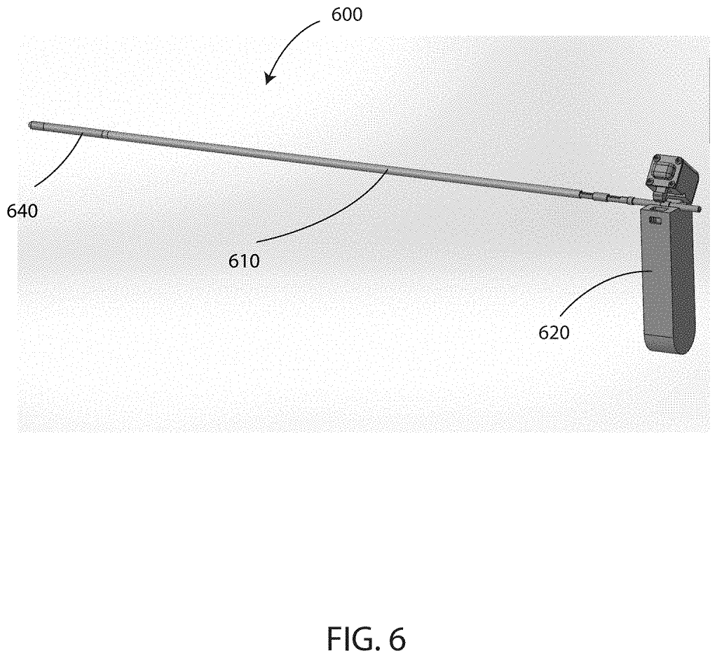

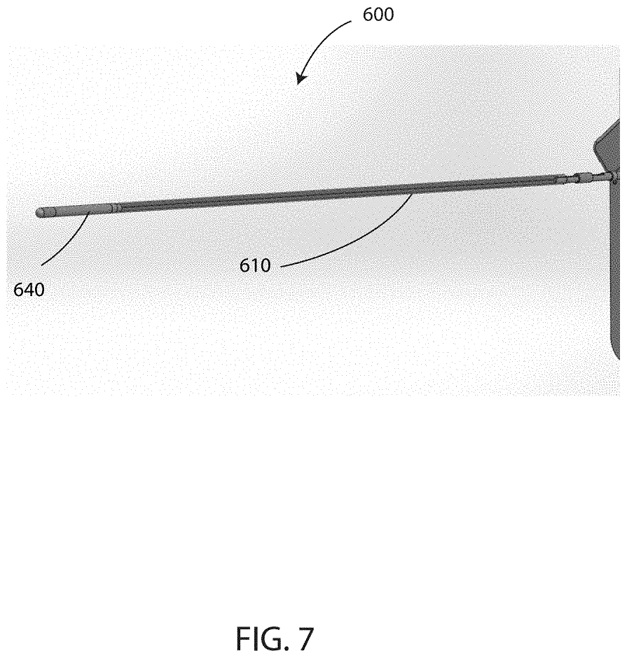

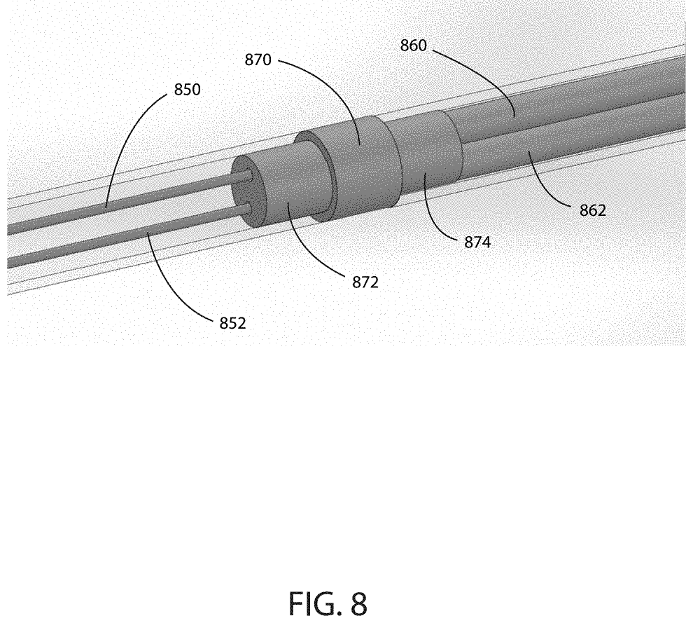

FIG. 6 is a drawing showing a laryngoscopy tool 600 for assisting in placement of an endotracheal tube made in accordance with an example embodiment. The laryngoscopy tool 600 includes a stylet 610, a handle 620 (at the proximal end of the tool), an actuator and a tip 640 at a distal end. The actuator 530 can be moved (such as sliding forward or backward, or by rotation) to engage an internal mechanism that causes the tip 640 of the stylet 610 to bend (articulate). FIG. 7 is a drawing showing a portion of a laryngoscopy tool 600 for assisting in placement of an endotracheal tube made in accordance with an example embodiment. The flexible portion is shown with upper control wire 850 and lower control wire 852. The upper control wire 850 is retained within the stylet 610 within an upper sheath 860; while the lower control wire 852 is retained within lower sheath 862. A transition segment 870 is shown, with a distal portion 872 and proximal portion 874. This transition segment separates the articulating tip (with upper control wire 850 and lower control wire 852 positioned to actuate flexing in the tip) from the less flexible stylet 610 portion. FIG. 8 is a drawing showing a transition segment of a laryngoscopy tool 600 that can transition from a stiffer main shaft to the more flexible articulation zone in the tip 640.

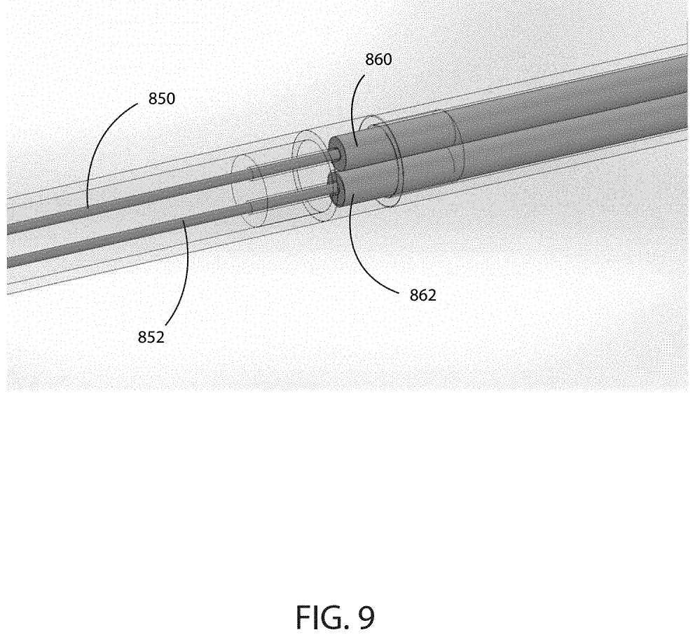

FIG. 9 is a partially-transparent image showing a transition segment of a laryngoscopy tool 600 of FIG. 6 that can transition from a stiffer main shaft to the more flexible articulation zone.

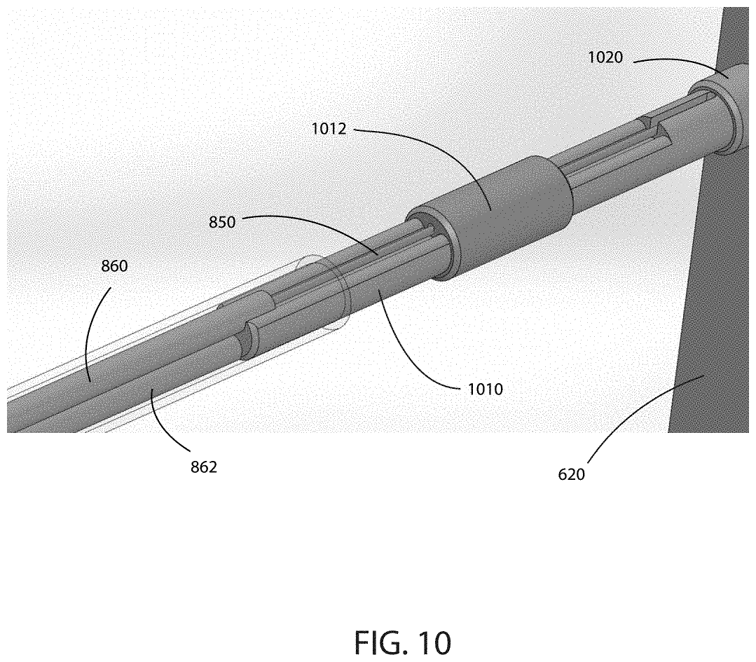

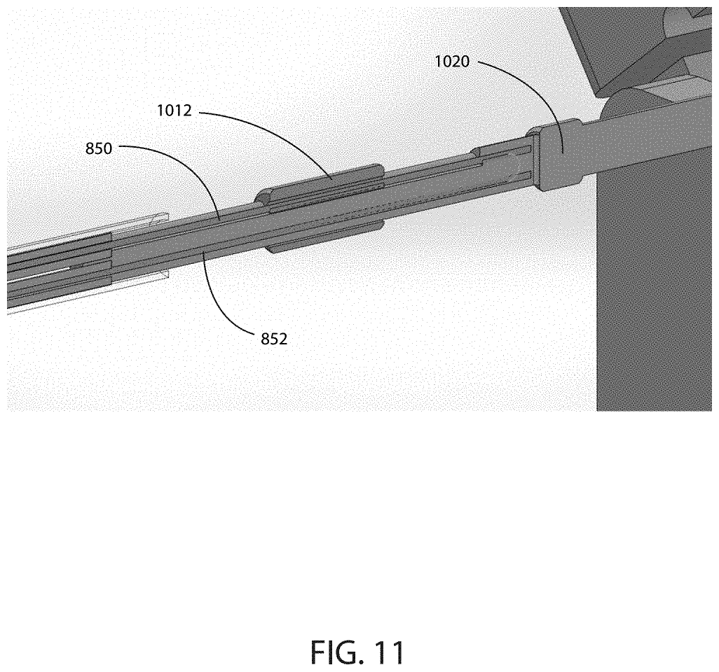

FIG. 10 is a partially-transparent image showing a further section of a laryngoscopy tool of FIG. 6. FIG. 11 is a partial cross section showing a further section of a laryngoscopy tool of FIG. 6. Here an extension 1010 from the handle 620 is shown, including a steering control base 1012 into which upper control wire 850 and lower control wire 852 (not shown) pass on way to connect at junction 1020 of the handle 620. The upper control wire 850 and lower control wire 852 can be made, for example, of a polymeric material, a metal material, etc. The control wires may be multi-strand or single strand in various embodiments.

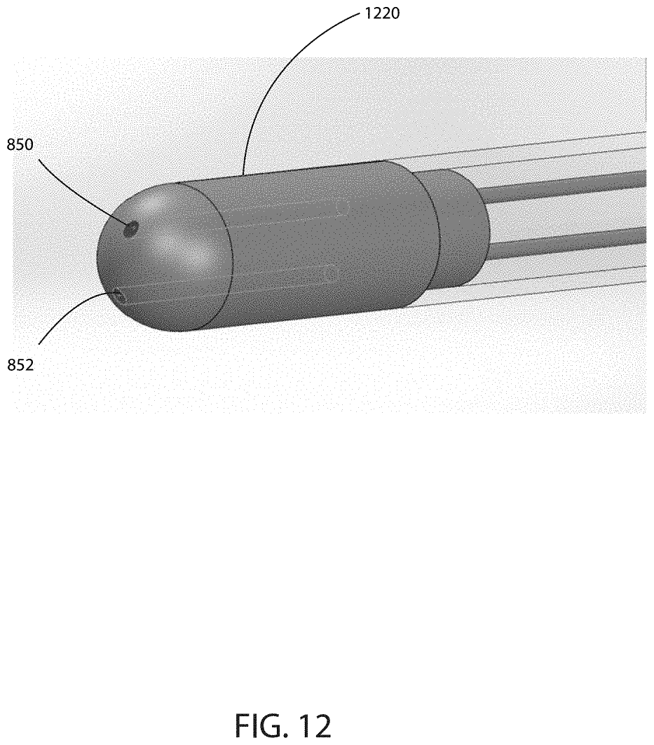

FIG. 12 is an example end 1220 of a laryngoscopy tool 600, in which the end 1220 has upper control wire 850 and lower control wire 852 extend through the end 1220 during manufacture, followed by being sealed in place and cut off.

The laryngoscopy tool can articulate using an apparatus such as those shown, for example, in U.S. Pat. No. 5,611,777 or 5,931,811, the contents of which are incorporated herein by reference. Also, the laryngoscopy tool can be of varying lengths suitable for use with patients of all sizes, from young children to adults.

It should be noted that, as used in this specification and the appended claims, the singular forms “a,” “an,” and “the” include plural referents unless the content clearly dictates otherwise. It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

We claim:

1. A laryngoscopy tool for assisting in placement of an endotracheal tube, the laryngoscopy tool comprising:
a) a stylet having a proximal end and a distal end, the stylet:
i.) configured for receiving an endotracheal tube, and
ii) having an articulating distal end, the articulating distal end being configured to articulate between more than one shape; and
b) a handle secured to the proximal end of the stylet.

2. The laryngoscopy tool for assisting in placement of an endotracheal tube of claim 1, wherein the handle is removable from the proximal end of the stylet.

3. The laryngoscopy tool for assisting in placement of an endotracheal tube of claim 1, wherein the handle is removable from the proximal end of the stylet to allow placement of an endocardial tube in place.

4. The laryngoscopy tool for assisting in placement of an endotracheal tube of claim 1, wherein the stylet articulates in a plane.

5. The laryngoscopy tool for assisting in placement of an endotracheal tube of claim 1, wherein the stylet includes an internal mechanism for articulating the distal end.

6. The laryngoscopy tool for assisting in placement of an endotracheal tube of claim 1, where the stylet further comprises a flexible intermediate portion.

7. The laryngoscopy tool for assisting in placement of an endotracheal tube of claim 1, wherein the stylet comprises an internal wire connected to the distal end, and wherein pulling on the wire from the proximal end results in articulating the distal end of the stylet.

8. The laryngoscopy tool for assisting in placement of an endotracheal tube of claim 1, further comprising an actuator.

9. The laryngoscopy tool for assisting in placement of an endotracheal tube of claim 8, wherein the actuator comprises a tube surrounding a portion of the proximal end of the stylet.

10. A method for placement of an endotracheal tube, the method comprising:
a) providing a laryngoscopy tool for assisting in placement of an endotracheal tube, the laryngoscopy tool comprising:
i) a stylet having a proximal end and a distal end, the stylet configured for receiving an endotracheal tube, and having an articulating distal end, the articulating distal end being configured to articulate between more than one shape; and
ii) a handle secured to the proximal end of the stylet;
b) inserting the distal end of the stylet of the laryngoscopy tool into the trachea of a person, including articulating the distal end of the stylet during insertion to aid in passage through the larynx;
c) removing the handle from the proximal end of the stylet;
d) inserting an endotracheal tube over the proximal end of the stylet and down toward the distal end of the stylet; and
e) removing the stylet from the patient's trachea will keeping the endotracheal tube in place.

11. The method for placement of an endotracheal tube of claim 10, wherein the laryngoscopy tool for assisting in placement of an endotracheal tube is removable from the proximal end of the stylet.

12. The method for placement of an endotracheal tube of claim 10, wherein the laryngoscopy wherein the stylet articulates in a plane.

13. The method for placement of an endotracheal tube of claim 10, wherein the laryngoscopy wherein the stylet includes an internal mechanism for articulating the distal end.

14. The method for placement of an endotracheal tube of claim 10, wherein the stylet further comprises a flexible intermediate portion.

15. The method for placement of an endotracheal tube of claim 10, wherein the stylet comprises an internal wire connected to the distal end, and wherein pulling on the wire from the proximal end results in articulating the distal end of the stylet.

16. The method for placement of an endotracheal tube of claim 10, further comprising an actuator.

17. The method for placement of an endotracheal tube of claim 10, wherein the actuator comprises a tube surrounding a portion of the proximal end of the stylet.
